(19) United States
(12) Patent Application Publication
(10) Pub. No.: US 2011/0046619 A1
(43) Pub. Date: Feb. 24, 2011

(54) Echogenic Electrosurgical Device

(75) Inventor: Richard W. Ducharme, Winston-Salem, NC (US)

Correspondence Address: BRINKS HOFER GILSON & LIONE/CHICAGO/COOK, PO BOX 10395, CHICAGO, IL, 60610, US

(73) Assignee: Wilson-Cook Medical Inc., Winston-Salem, NC (US)

(21) Appl. No.: 12/858,936

(22) Filed: Aug. 18, 2010

Related U.S. Application Data
(60) Provisional application No. 61/235,115, filed on Aug. 19, 2009.

(51) Int. Cl. A61B 18/14

(52) U.S. Cl. 606/41

(57) ABSTRACT

An echogenic electrosurgical device and a method for electrosurgically treating a target site are provided. The device includes an elongate body having a proximal portion and a distal portion. The distal portion of the elongate body includes an echogenic region, a coated portion providing an electroinsulative layer and an uncoated electroconductive electrosurgical region. The coating allows reflection of ultrasonic waves from the coated echogenic region sufficient for ultrasonic imaging of the echogenic region at a resolution providing for effective navigation in a body. The coated region has a first surface area and the electrosurgical region has a second surface area. The first surface area is greater than the second surface area.

### CROSS-REFERENCE TO RELATED APPLICATIONS

This application claims the benefit of U.S. Provisional Application No. 61/235,115, filed Aug. 19, 2009, which is incorporated herein by reference in its entirety.

### TECHNICAL FIELD

This invention generally relates to devices and methods for ultrasonically visualizable devices and more particularly to ultrasonically visualizable devices for electrosurgically treating tissue.

### BACKGROUND

The ability to monitor the location and orientation of surgical instrumentation within intraluminal and extraluminal regions of a patient is critical. Fluoroscopy and radiopaque materials have traditionally been used to create visible regions of the digestive tract. Fluoroscopy is a technique in which an x-ray beam is transmitted through a patient to generate images of the gastrointestinal (GI) lumen that appear on a television monitor. It can also be used to observe the action of instruments during diagnostic procedures. However, x-rays consist of electromagnetic radiation which can be dangerous to the bile duct and pancreatic duct.

Conventional endoscopy offers visualization of the intraluminal regions through which the endoscope is inserted due to a video camera attached at the distal end of the endoscope. However, the video camera provides a field of view limited to only the intraluminal region. The use of surgical instrumentation outside of the lumen into extraluminal regions cannot be visualized with the endoscopic video camera.

Medical ultrasound has been another option used to monitor instrumentation. Medical ultrasound utilizes high frequency sound waves to create an image of living tissue. As ultrasound waves are emitted, the waves reflect when encountering a surface change. The reflected waves are used to create an image. Ultrasound allows for monitoring of the medical devices in extraluminal regions as well as in intraluminal regions. Such monitoring is necessary to ensure medical devices are guided to their target sites and not inadvertently damaging adjacent tissue.

Various types of medical devices are commonly used for diagnostic and therapeutic gastrointestinal endoscopy and provide access to the digestive tract. Common endoscopy procedures include incision, sampling and ablation of various tissues. Multiple devices may be used to perform a single procedure, such as a cutting device and a cautery device. In one exemplary procedure, a cutting device may be used within the gastrointestinal tract for removing part of the digestive wall including the mucosal membrane, i.e. endoscopic mucosectomy. These cutting procedures can cause bleeding and trauma to the tissue and increase patient healing time. It is important to reduce the amount of trauma to the patient as well as the length of the procedure. Therefore, it is beneficial to have a device that can be closely monitored while performing the procedure as well having a device that can perform both a cutting function and a cautery function at the target site.

### SUMMARY OF THE INVENTION

Accordingly, it is an object of the present invention to provide a device that is echogenic and that also performs an electrosurgical procedure at a target site.

The foregoing object is obtained in one aspect of the present invention by providing an echogenic electrosurgical device. The device includes an elongate body having a proximal portion and a distal portion. The distal portion of the elongate body includes an echogenic region, a coated portion providing an electroinsulative layer and an uncoated electorconductive electrosurgical region. The coating allows reflection of ultrasonic waves from the coated echogenic region sufficient for ultrasonic imaging of the echogenic region at a resolution providing for effective navigation in a body. The coated region has a first surface area and the electrosurgical region has a second surface area. The first surface area is greater than the second surface area.

In another aspect of the present invention, an echogenic electrosurgical system is provided. The system includes an outer sheath having a proximal portion and a distal portion and a lumen extending at least partially therethrough. The system also includes an elongate body positonable at least partially within the lumen including a proximal portion and a distal portion. The body also includes an electroconductive material. The body distal portion includes an echogenic region, a coated portion including a coating on at least a portion of the echogenic region, the coating providing an electroinsulative layer on the coated portion that allows reflection of ultrasonic waves from the coated echogenic region sufficient for ultrasonic imaging of the echogenic region at a resolution providing for effective navigation in a body, and an uncoated, electroconductive electrosurgical region. The system further includes a handle including an electrode operably connected to the elongate body.

In another aspect of the present invention, a method for providing an ultrasonically guided electrosurgical device to a target site in a patient is provided. The method includes providing an elongate body having a proximal portion and a distal portion, and including an electroconductive material. The distal portion of the body includes an echogenic region, a coated portion including a coating on at least a portion of the echogenic region, the coating providing an electroinsulative layer on the coated portion that allows reflection of ultrasonic waves from the coated echogenic region sufficient for ultrasonic imaging of the echogenic region at a resolution providing for effective navigation in a body and an uncoated, electroconductive electrosurgical region. The method also includes directing the distal portion to the target site using ultrasound visualization of the echogenic region, supplying an electrical current to elongate body, contacting the target site with the electrosurgical region and electrosurgically treating the tissue.

### BRIEF DESCRIPTION OF THE DRAWINGS

FIG. 1 is a partial side view of an embodiment of an echogenic electrosurgical device according to the present invention;

FIG. 2 is a partial side view of another embodiment of an echogenic electrosurgical device according to the present invention;

FIG. 3 is a partial side view of another embodiment of an echogenic electrosurgical device according to the present invention;

FIG. 4 is a side view of an embodiment of an echogenic electrosurgical device according to the present invention showing a handle; and

FIG. 5 is a diagrammatic view of an echogenic electrosurgical device within the GI tract for treatment of a tissue.

### DETAILED DESCRIPTION OF THE EMBODIMENTS

The invention is described with reference to the drawings in which like elements are referred to by like numerals. The relationship and functioning of the various elements of this invention are better understood by the following detailed description. However, the embodiments of this invention are not limited to the embodiments illustrated in the drawings. It should be understood that the drawings are not to scale, and in certain instances details have been omitted which are not necessary for an understanding of the present invention, such as conventional fabrication and assembly.

As used in the specification, the terms proximal and distal should be understood as being in the terms of a physician delivering the device to a patient. Hence the term “distal” means the portion of the echogenic cutting and cautery device that is farthest from the physician and the term “proximal” means the portion of the device that is nearest to the physician.

As used herein, the term “echogenic” is defined as having enhanced echogenicity. Specifically, it is used to refer to materials or portions of materials that are constructed or are treated to have greater reflectivity of ultrasonic waves than standard materials used for a sheath, cannula, catheter, and/or stylet, and to provide an echogenic profile relative to surrounding tissues during use in a patient body to accurately orient and direct the echogenic device portion. It is known in the art that most materials used for a sheath, catheter, cannula, or stylet will reflect some ultrasonic waves, but the term “echogenicity,” as used herein includes treating the surface by creating a textured or patterned surface including, for example, one or more of dimples, divots, knurling, ridges, or the like—each of which is known in the art to enhance echogenicity as compared to a smooth surface for a similarly-sized/shaped object, (and/or, when specifically referenced, using a material known to provide an enhanced echogenic profile) configured to provide clear ultrasound visualization at a resolution providing for accurate location and navigation of a device in a body (e.g., of a patient).

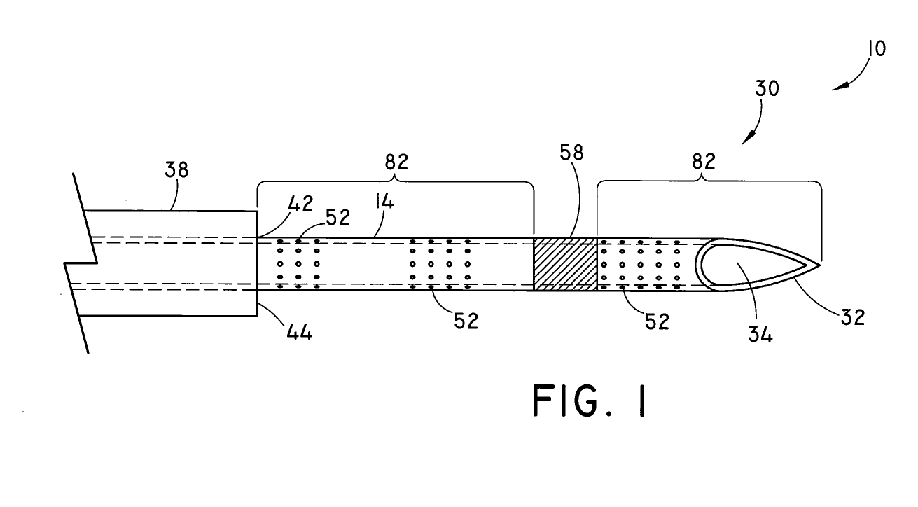

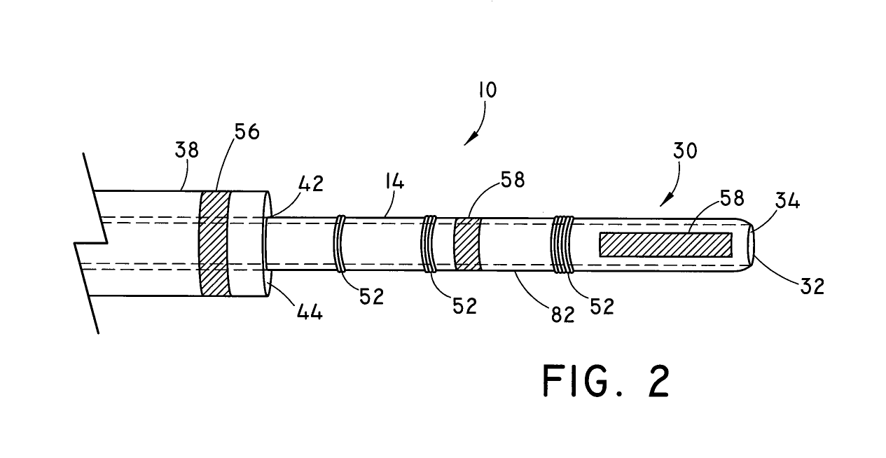

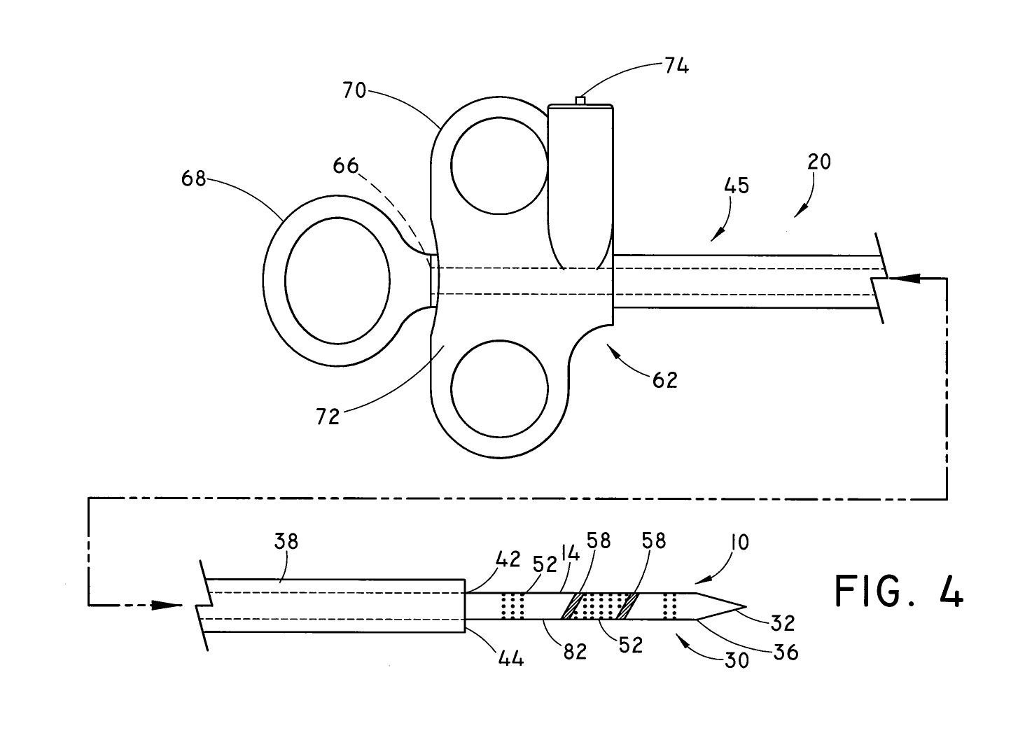

FIGS. 1 and 2 illustrate an echogenic electrosurgical device 10 in accordance with embodiments of the present invention. The device 10 includes a generally elongate body 14 having a proximal portion 20 (shown in FIG. 4) and a distal portion 30. The distal portion 30 includes a tip 32 that may be used for penetrating through an occlusion, a stomach wall, an intestinal wall, or another artificial or natural structure between an endoscopically accessible site and a target site, including the creation of an orifice for a natural orifice translumenal endoscope (NOTES). The tip 32 may be pointed as shown in FIG. 1, beveled or blunt (as shown in FIG. 2). The tip 32 may also include a sharp surface 36 extending longitudinally in the form of a blade for cutting. (See FIG. 4.) The body 14 may include a lumen 34 extending at least partially through the body 14 as shown in FIGS. 1 and 2. Alternatively, the body 14 may be solid as shown in FIG. 4.

The device 10 may further include an outer sheath 38 having a lumen 42 extending at least partially therethrough. The body 14 may be provided within the lumen 42 and slidable relative to the sheath 38 so that the distal tip 32 of the body 14 may be extended distally from a distal end 44 of the sheath 38 for insertion into the target tissue. The distal tip 32 may be protected within the lumen 42 of the sheath 38 until the distal end 44 of the sheath 38 near the target site within the patient.

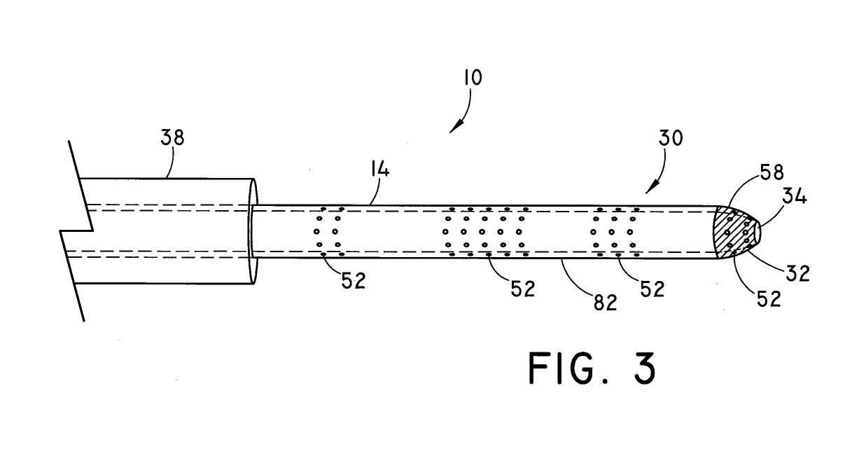

The body 14 includes one or more echogenic regions 52 on the distal portion 30. The body 14 also includes one or more electrosurgical regions 58 on the distal portion 30. The electrosurgical region 58 may be used to cauterize, ablate, cut or otherwise eletrosurgically treat the tissue. The electrosurgical regions 58 may be made by leaving the electrosurgical regions 58 uncoated and coating the remaining portions of the distal portion 30 of the body 14 that would potentially contact the tissue. The echogenic regions 52 may be positioned on the distal portion 30 of the body adjacent to, at least partially overlapping or at a distance from the electrosurgical region 58 to indicate the location of the electrosurgical regions 58 during a procedure. The echogenic regions 52 and the electrosurgical regions 58 may be in any shape and size. For example, the electrosurgical region 58 may be provided as a ring encircling the body 14 and having a width that is less than the length of the distal portion 30 that is exposed to the tissue. In some embodiments, the electrosurgical region 58 is formed in a ring encircling the body 14 and is spaced apart from the tip 34 as shown in FIG. 1. In some embodiments, the surface area of the electrosurgical regions 58 may be 10% or less of the length of the distal end 30 that is exposed to the tissue so that the energy at the contact point with the tissue is not dissipated. Alternatively or in addition, the electrosurgical region 58 may be in the shape of a longitudinally extending rectangle or zigzag or other shape only on a portion of the circumference of the body 14 as shown in FIG. 2. The electrosurgical region 58 may also be provided at the distal tip 34 and having a size greater than only a point as shown in FIG. 3. The echogenic region 52 may also be at the same position on the body 14 as the electrosurgical region 58.

As shown in FIG. 4, the body 14 and sheath 38 may be provided with a handle assembly 62 attached to a proximal portion 45 of the sheath 38 and a port 66 through a housing 72. Additional and components may be added to the handle assembly 62 depending on the intended procedure. For example, one or more additional ports may also be included, such as an aspiration port or an irrigation port. As shown, the handle assembly 62 includes two portions, a first portion 68 and a second portion 70 that are moveable with respect to each other. The housing 72 of the handle assembly 62 also includes one or more electrodes 74 that are connectable to an electrosurgical generator (not shown). The electrode 74 is in contact with a portion of the body 14. Additional handle assemblies may be used with body 14 and sheath 38 as will be understood by one skilled in the art. For example, the handle assembly may use a different number of rings, a trigger grip, or other gripping surfaces to help manipulate the body 14 position within the patient.

The electrosurgical portion 58 of the body 14 has a surface area that is uncoated and is surrounded by coated portions having a greater surface area than the uncoated surface area to insulate the body 14 and to target the energy to the electrosurgical portion 58. For example, when the electrosurgical region 58 is provided as a ring encircling the body 14 and spaced apart from the distal tip 34, the coated portion of the body 15 keeps the current higher around the uncoated electrosurgical region 58 so that a concentric ring of ablated tissue may be formed. A portion of the body 14 is coated with a polymeric coating 82. For clarity, the polymeric coating 82 is indicated in FIG. 1 to cover the distal portion of the body 14 with the exception of the electrosurgical portion 58. Similarly, in FIGS. 2-4, it should be understood that the coating 82 covers the distal portion 30 of the body 14 with the exception of the electrosurgical portions 58. The polymeric coating 82 covers at least one or more of the electrosurgical portions 52 so the coating must be thin enough to not interfere with the visualization of the echogenic portions 52 during a procedure. In some embodiments, the coating 82 may be between a fraction of a micron and several thousandths of an inch in thickness. In some embodiments, the thickness of the coating 82 may be between about 5 μm to about 50 μm. The coating may also have a low coefficient of friction and is insulative of the electrical current applied to the body 14. The coating 82 provides insulation for the body 14 in the coated portions, exclusive of the electrosurgical regions 58, when electric current is passed from the electrode 74 to the body 14 and also provides viewability of the echogenic portions 52. The coating 82 allows the current density at the point of tissue contact with the electrosurgical region 58 to remain constant and provide for precise cutting or cautery. For example, when the electrosurgical region 52 is provided in the shape of a ring encircling the body 14, it is possible to cauterize a band of tissue or to cut a concentric hole through the tissue without needing to contact the tissue on a point by point basis to form the band. In some embodiments, a band of about 2 mm may form the electrosurgical region 58 with the remaining portion of the distal portion 30 coated with the coating 82.

In some embodiments, the coating 82 may be made from parylene-N (poly-p-xylylene). Other xylylene polymers, and particularly parylene polymers, may also be used as a coating within the scope of the present invention, including, for example, 2-chloro-p-xylylene (Parylene C), 2, 4-dichloro-p-xylylene (Parylene D), poly(tetraflouro-p-xylylene), poly(carboxyl-p-xylylene-co-p-xylylene), fluorinated parylene, or parylene HT® (a copolymer of per-fluorinated parylene and non-fluorinated parylene), alone or in any combination. Preferred coatings of the present will include the following properties: low coefficient of friction (preferably below about 0.5, more preferably below about 0.4, and most preferably below about 0.35); very low permeability to moisture and gases; fungal and bacterial resistance; high tensile and yield strength; high conformality (ready application in uniform thickness on all surfaces, including irregular surfaces, without leaving voids); radiation resistance (no adverse reaction under fluoroscopy); bio-compatible/bio-inert; acid and base resistant (little or no damage by acidic or caustic fluids); ability to be applied by chemical vapor deposition bonding/integrating to wire surface (bonding is intended to contrast to, for example, fluoroethylenes that form surface films that are able to be peeled off an underlying wire); and high dielectric strength. Parylene coatings, in particular, exhibit these qualities. See, for example, Table 1.

TABLE 1
Typical Parylene Properties

| | | | |
|---|---|---|---|
| Parylene | Parylene | Parylene | Parylene |
| N | C | D | HT |

Typical Physical & Mechanical Properties

| | | | | | |
|---|---|---|---|---|---|
| Coefficient | static | 0.25 | 0.29 | 0.33 | 0.145 |
| of friction: | dynamic | 0.25 | 0.29 | 0.31 | 0.13 |

| | | | | |
|---|---|---|---|---|
| Melt point (° C.) | 420 | 290 | 380 | 7500 |

Typical Electrical Properties

| Dielectric strength, | | 7,000 | 5,600 | 5,500 | 5,400 |
|---|---|---|---|---|---|
| short time | | | | | |
| (Volts/mil at 1 mil) | | | | | |
| Di- | 60 Hz | 2.65 | 3.15 | 2.84 | 2.21 |
| electric | 1,000 Hz | 2.65 | 3.1 | 2.82 | 2.2 |
| con- | 1,000,000 Hz | 2.65 | 2.95 | 2.8 | 2.17 |
| stant: | | | | | |

The coating 82 may be applied to the body 14 according to any coating procedure known to one skilled in the art that can provide a coating in a thickness suitable for viewing the echogenic regions 52 and to insulate the body 14. The coating 82 may be applied to the body 14 by chemical vapor deposition (“CVD”, which may include a plasma-assisted CVD process). Chemical vapor deposition is a well-known process in the art of electronic circuitry that is well-adapted for applying a coating, such as—for example—a parylene coating, to a device. The process smoothly and uniformly applies the coating to the device around its circumferential surface. A coated body 14 using a parylene coating presents advantages in coating durability, cost savings, and desirable outer diameter, while providing a coating with excellent lubricity (low friction) and electroinsulative qualities. In contrast with prior art coatings, a bonded coating of the present invention will not split or peel away from the wire due to frictional or traumatic contact with another surface such as, for example, an endoscope or the outer sheath. The body 14 of the present invention is electroconductive and may be constructed of stainless steel, nitinol, or another electroconductive material within the scope of the present invention.

The electrosurgical regions 58 of the body 14 may be coated with a removable protective masking application of the coating 82 to the body 14. The removable protective masking is provided to protect the electrosurgical region 58 during the insulative coating application process. The removable protective masking is removed after the coating 82 has been applied to the body 14.

Coating the body in the targeted fashion of the described embodiment of a method will provide a desired electroinsulative coating, while also providing a minimal use of the electroinsulative coating and attendant cost savings. The thinness and uniformity of the coating, whether applied by chemical vapor deposition or another process preferably are consistent along the coated body length, but most preferably provide an integrity-maintaining coating in a region of the body that is to be exposed outside the sheath but not intended to be used for cutting (specifically that region of the body immediately adjacent the electrosurgical region and exposed outside the sheath during normal operation).

The power source for the electrical supply may be any suitable source for delivering power for a surgical procedure. The electrical supply may be monopolar or bipolar. For example, a bipolar device may be provided by including a small insulated return wire connected to a portion of the needle that is electrically insulated from the needle (not shown).

The echogenic region 52 may be formed using any method known to one skilled in the art. By way of non-limiting example, dimples, grooves or ridges may be provided randomly on the surface of the body 14, or in more regular patterns, for example in geometric shapes and patterns such as concentric circles, or as lines running substantially parallel or perpendicular to an axis of the device e.g. in a circumferential arrangement to give bands or corsets, or in a helical arrangement. Suitable patterns can be readily determined to suit the exact size and shape of the medical device concerned. Patterns may also be provided to help the physician monitor the location of the device 10 relative to the target tissue and to ensure that the body 14 remains in the field of view of the ultrasonic scanning plane if incident ultrasound wave inadvertently do not strike the distal-most echogenic region 52. The patterns may also help the physician identify additional functional portions of the body may be located, for example, a cautery region, as described below. The dimpled, grooved or ridged surface may also be achieved by etching, for example using a laser or water-jet cutter, electrolytic etching or by blasting, such as sand blasting. Exemplary echogenic devices and methods may also be found in U.S. Publication Number 2008/0097213, which is incorporated by reference in its entirety. One example of a device having an echogenic region on a body and sheath surrounding the body may be found in the EchoTip® needle (available from Cook Medical, Bloomington, Ind.).

In some embodiments, the outer sheath 38 may also be provided with one or more echogenic regions 56. The echogenic regions 56 may be formed using the methods described above or any method known to one skilled in the art. By way of non-limiting example, the outer sheath 38 may be formed from stainless steel, i.e. a hypotube, or a nickel-titanium alloy. In some embodiments, the outer sheath may be formed from a polyether block amide (PEBA), polyetheteher ketone (PEEK), ePTFE, PTFE, or PET materials.

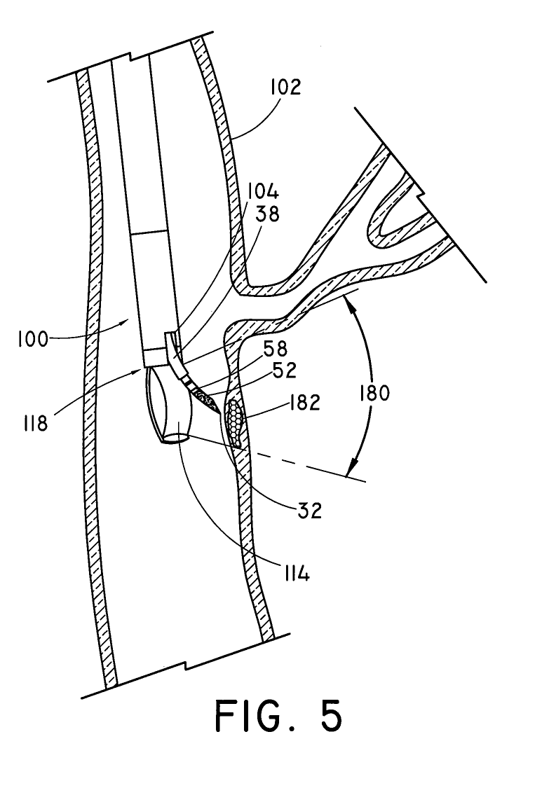

An exemplary method of treating a tissue with the echogenic electrosurgical device 10 is shown in FIG. 5. Typically an endoscope or an endoscopic ultrasound (EUS) device that utilizes high frequency sound waves to create an image of living tissue or an echogenic surface, is positioned in the duodenum 102. An EUS device 100 is shown in FIG. 5 having an ultrasonic array of transducers 114 at the distal end 118 of the endoscope 100. The transducers 114 may be connected to an imaging system (not shown) for viewing the image created by the ultrasonic transducers 114 and the device 10 with the echogenic region 52. The transducers 114 generate an ultrasonic scanning plane 180 to permit real-time monitoring of the medical device location and orientation within the scanning plane 180. The body 14 and the sheath 38 of the device 10 are shown extending from an accessory channel 104 the EUS device 100 and directed to a target tissue 182 in the wall of the duodenum 102. The distal tip 32 of the body 14 may be inserted into the tissue at the target treatment site 182 using the image from the ultrasonic transducers 114 to position the tip of the body 14 in the correct location. A tissue sample may be taken through the lumen 34 of the body 14 if desired. The electrosurgical region 58 may be advanced to the tissue and the electric supply initiated to cauterize the tissue in a circular band surrounding the entry point of the distal tip 32 using the electrosurgical region 58 against the tissue. The surrounding tissue will be unaffected due to the coating 82 on the distal portion of the body 14. Similarly, the electrosurgical region 58 may be used to cut the tissue at the target site 182 by supplying the electric current to the body 14 so that the electrosurgical region 58 cuts into the tissue only at the electrosurgical region 58, for example when a rounded distal tip 32 is provided on the body 14. When the electrosurgical region 58 is provided as a ring encircling the body 14, a concentric burn of target tissue is achieved away from the tip 34. Having the coating 82 allows the current concentration to be higher at the ringed electrosurgical region 58 and not at the tip 34 when the tip 34 is coated (See FIG. 1). Without the coating 82, the current density drops and the cautery occurs at the tip of the needle. The ringed electrosurgical region 58 allows for cautery of a punctured vascular organ to stop or slow bleeding around a circular puncture wound with concentrated current to provide enough energy for a concentric burn.

The above Figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims. For example, the invention has been described in the context of the biliary system for illustrative purposes only. Application of the principles of the invention to any other bifurcated lumens or vessels within the body of a patient, including areas within the digestive tract such as the pancreatic system, as well as areas outside the digestive tract such as other vascular systems, by way of non-limiting examples, are within the ordinary skill in the art and are intended to be encompassed within the scope of the attached claims.

1. An echogenic electrosurgical device, the device comprising:
an elongate body having a proximal portion and a distal portion, the distal portion including a distal tip, the distal portion comprising:
an echogenic region;
a coated portion having a first surface area, the coated portion including a coating on at least a portion of the echogenic region, the coating providing an electroinsulative layer on the coated portion that allows reflection of ultrasonic waves from the coated echogenic region sufficient for ultrasonic imaging of the echogenic region at a resolution providing for effective navigation in a body; and
an uncoated, electroconductive electrosurgical region, the electrosurgical region having a second surface area;
wherein the first surface area is greater than the second surface area.

2. The device of claim 1, wherein the echogenic region and the electrosurgical region at least partially overlap.

3. The device of claim 1, wherein the distal portion includes a plurality of echogenic regions.

4. The device of claim 1, wherein the electrosurgical region comprises a circumferential band on the distal portion.

5. The device of claim 4, wherein the band extends about 2 mm along a longitudinal axis of the elongate body.

6. The device of claim 1, wherein the electrosurgical region comprises a longitudinally extending portion that does not extend circumferentially around the elongate body.

7. The device of claim 1, wherein the distal tip is blunt or pointed.

8. The device of claim 1, wherein the coating is selected from the group consisting of poly-p-xylylene, 2-chloro-p-xylylene, 2, 4-dichloro-p-xylylene, poly(tetraflouro-p-xylylene), poly(carboxyl-p-xylylene-co-p-xylylene), fluorinated parylene, or a copolymer of per-fluorinated parylene and non-fluorinated parylene, and any combination thereof.

9. The device of claim 1, wherein the coating comprises a polymeric coating material having a static coefficient of friction below about 0.4.

10. The device of claim 1, wherein the coating thickness is between about 5 μm to about 50 μm.

11. The device of claim 1, wherein the elongate body comprises a lumen extending at least partially therethrough.

12. The device of claim 1, further comprising a handle operably connected to the elongate body, the handle comprising an electrode connected to an electrosugurical generator to provide electrical current to the electrosurgical region.

13. An echogenic electrosurgical system, the system comprising:
an outer sheath having a proximal portion and a distal portion and a lumen extending at least partially therethrough;
an elongate body positonable at least partially within the lumen, the elongate body having a proximal portion and a distal portion, the body comprising an electroconductive material, the body distal portion comprising:
an echogenic region;
a coated portion including a coating on at least a portion of the echogenic region, the coating providing an electroinsulative layer on the coated portion that allows reflection of ultrasonic waves from the coated echogenic region sufficient for ultrasonic imaging of the echogenic region at a resolution providing for effective navigation in a body; and
an uncoated, electroconductive electrosurgical region;
a handle including an electrode, the electrode operably connected to the elongate body.

14. The system of claim 13, wherein the outer sheath comprises an echogenic region.

15. The system of claim 13, wherein a distal end portion of the body comprises the electrosurgical region.

16. A method for providing an ultrasonically guided electrosurgical device to a target site in a patient; the method comprising:
providing an elongate body having a proximal portion and a distal portion, the body comprising an electroconductive material, the body distal portion comprising:
an echogenic region;
a coated portion including a coating on at least a portion of the echogenic region, the coating providing an electroinsulative layer on the coated portion that allows reflection of ultrasonic waves from the coated echogenic region sufficient for ultrasonic imaging of the echogenic region at a resolution providing for effective navigation in a body; and
an uncoated, electroconductive electrosurgical region
directing the distal portion to the target site using ultrasound visualization of the echogenic region;
supplying an electrical current to elongate body;
contacting the target site with the electrosurgical region; and
electrosurgically treating the tissue.

17. The method of claim 16, further comprising providing an outer sheath over the elongate body for delivering the elongate body to the target site.

18. The method of claim 17, comprising extending the elongate body distally of the outer sheath to expose the elongate body to the target site.

19. The method of claim 17, comprising providing the coating selected from the group consisting of poly-p-xylylene, 2-chloro-p-xylylene, 2, 4-dichloro-p-xylylene, poly(tetraflouro-p-xylylene), poly(carboxyl-p-xylylene-co-p-xylylene), fluorinated parylene, or a copolymer of per-fluorinated parylene and non-fluorinated parylene, and any combination thereof, to electroinsulate the elongate body.

20. The method of claim 16, comprising repositioning the distal portion at a second target site using ultrasound visualization of the echogenic region.
